(19) United States
(12) Patent Application Publication
(10) Pub. No.: US 2016/0015845 A1
(43) Pub. Date: Jan. 21, 2016

(54) OXIDIZING AGENT APPLICATION SYSTEM AND METHOD

(71) Applicant: MOJACK DISTRIBUTORS, LLC, Wichita, KS (US)

(72) Inventor: Buddy Don Gray, Dellrose, TN (US)

(73) Assignee: MOJACK DISTRIBUTORS, LLC, Wichita, KS (US)

(21) Appl. No.: 14/802,707

(22) Filed: Jul. 17, 2015

Related U.S. Application Data
(60) Provisional application No. 62/026,308, filed on Jul. 18, 2014.

(51) Int. Cl. A61L 2/18

(52) U.S. Cl. CPC A61L 2/183

(57) ABSTRACT

An oxidizing agent generating device and a liquid application device combine a gaseous oxidizing agent with a liquid creating an oxidizing agent-liquid material that is applied to a surface to incapacitate and destroy pathogens on the surface. The oxidizing agent ozone is combined with the liquid and the mixture is applied under pressure to the surface to render the pathogens ineffective. A surfactant is added to the mixture decreasing the tension of the mixture when it is in contact with the surface increasing the amount of dissolved ozone in contact with the surface.

### CROSS-REFERENCE TO RELATED APPLICATIONS

This application claims priority in U.S. Provisional Application No. 62/026,308, filed Jul. 18, 2014, the contents of which are hereby incorporated by reference.

### BACKGROUND OF THE INVENTION

1. Field of the Disclosed Subject Matter

The present disclosed subject matter relates generally to oxidizing agent application systems and methods, and more particularly to a mobile device for applying ozone dissolved in a liquid on a surface.

2. Background

Oxidizing agents can be used to incapacitate or destroy pathogens. A strong oxidizing agent is ozone (O₃). Ozone occurs naturally in the environment when an electrical discharge, such as lighting, passes through air containing the gaseous form of oxygen (O₂). Pathogens harmful to humans include microorganisms, such as fungus, protozoan, bacteria, and viruses. Contact of an oxidizing agent with a pathogen can render the pathogen ineffective. Ozone is reactive fur only a short time after it is generated, thus use of ozone as a disinfectant has limited residual harmful effects.

A liquid containing a dissolved oxidizing agent can be applied to a surface to incapacitate or destroy the pathogens thereon.

### SUMMARY OF THE INVENTION

An oxidizing agent application system mixes gaseous ozone with a liquid in a concentration sufficient to incapacitate and destroy pathogens on a surface. Gaseous ozone is combined with the liquid in a nozzle by delivering the ozone to the nozzle at a higher pressure than the liquid, creating an ozone-liquid mixture. The ozone-liquid mixture exits the nozzle under pressure and is applied to the surface.

An application device including an application system mounted to a mobile device, such as a trailer, applies the ozone-liquid mixture to a surface, such as a natural or artificial turf. The application system includes an oxidizing agent system, and a liquid system. The oxidizing agent system uses an ozone generator to create gaseous ozone and feeds the gaseous ozone to the nozzles. The liquid system moves the liquid to the nozzles that are attached in an array to a support structure located above the surface. The nozzles are where the gaseous ozone is combined with the liquid creating the ozone-liquid mixture with gaseous ozone dissolved in the liquid. Dissolving the ozone in a liquid, such as water, allows the dissolved ozone to remain in contact with the surface and any pathogens thereon, oxidizing the pathogens, and rendering any pathogens thereon ineffective.

A covering may be connected to the support structure to direct the ozone-liquid mixture and any undissolved gaseous ozone to the surface, and aid in retaining any undissolved gaseous ozone with the liquid on the surface to replace the ozone that is consumed in the oxidative process.

A surfactant or wetting agent may be added to the liquid and gaseous ozone to create an ozone-surfactant-liquid material. The addition of a surfactant to the liquid decreases the tension of the resulting mixture thereby increasing the amount of the ozone-liquid mixture in contact with the surface and the dissolved ozone in contact with any pathogens thereon.

### DESCRIPTION OF THE DRAWINGS

The drawings constitute a part of this specification and include exemplary embodiments of the disclosed subject matter and illustrate various objects and features thereof.

FIG. 1 is a rear perspective view of an application system embodying principles of the disclosed subject matter mounted to a trailer.

FIG. 2 is a schematic of the application system.

FIG. 3 is an enlarged view of the nozzle.

FIG. 4 is a rear elevation view of the an application system embodying principles of the disclosed subject matter mounted to a trailer.

FIG. 5 is a side elevation view of the an application system embodying principles of the disclosed subject matter mounted to a trailer.

FIG. 6 is a front perspective view of the an application system embodying principles of the disclosed subject matter mounted to a trailer.

FIG. 7 is a rear perspective view of an application system embodying principles of the disclosed subject matter employing a covering.

### DETAILED DESCRIPTION OF THE INVENTION

As required, detailed aspects of the disclosed subject matter are disclosed herein; however, it is to be understood that the disclosed aspects are merely exemplary of the disclosed subject matter, which may be embodied in various forms. Therefore, specific structural and functional details disclosed herein are not to be interpreted as limiting, but merely as a basis for the claims and as a representative basis for teaching one skilled in the art how to variously employ the disclosed technology in virtually any appropriately detailed structure.

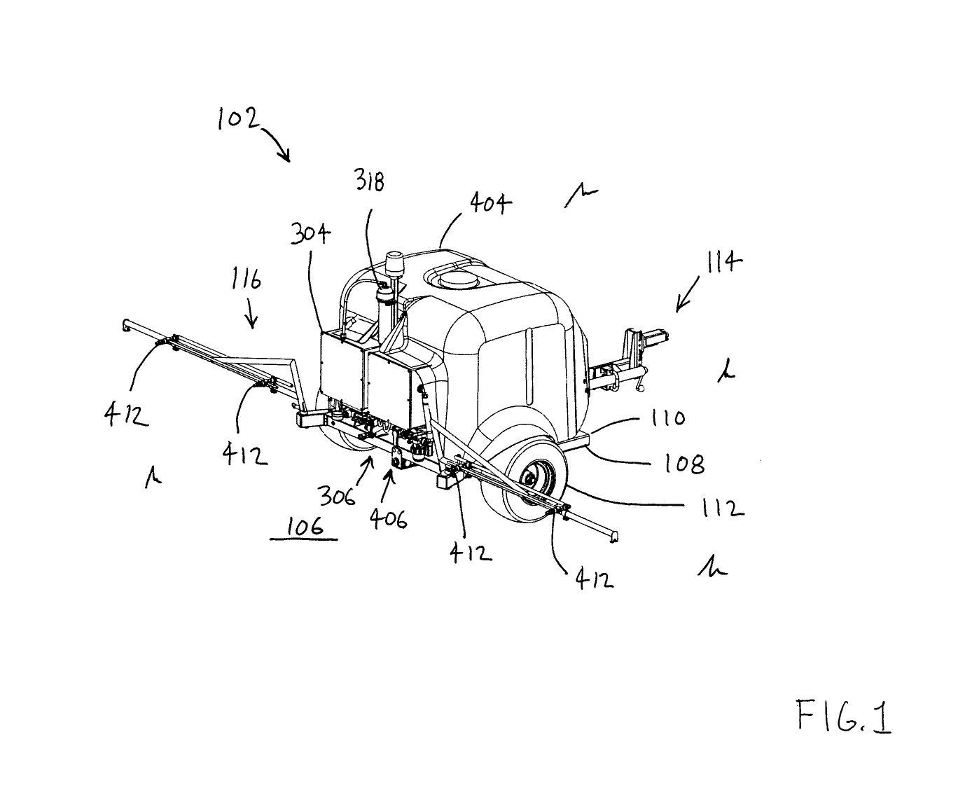

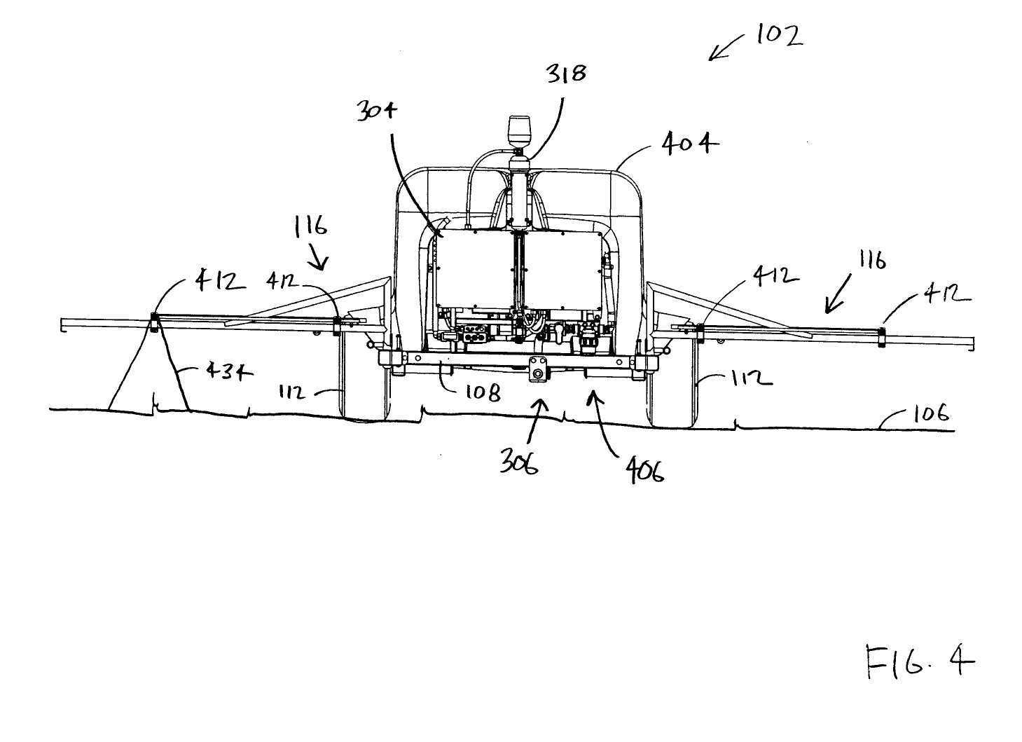

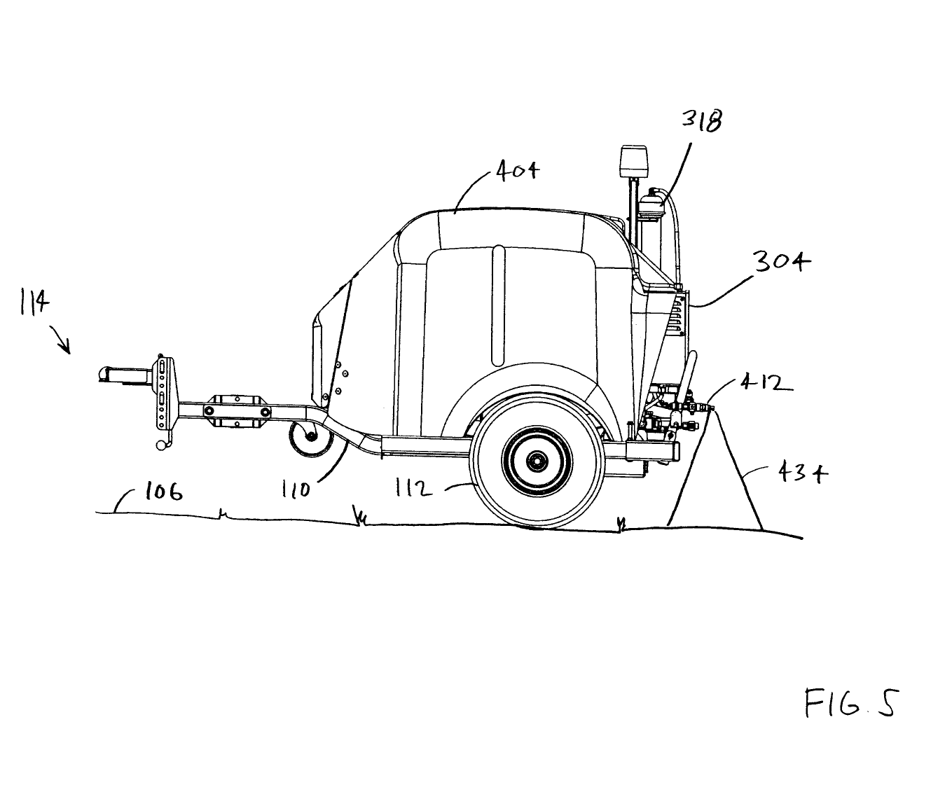

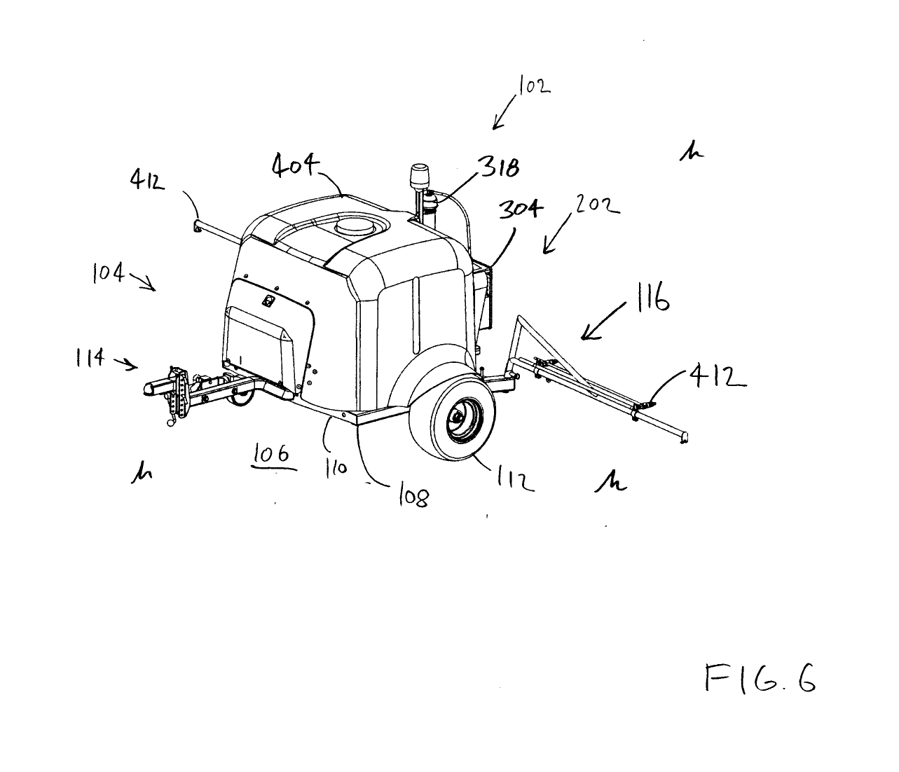

An application device 102 including an application system 202 mounted to a mobile device 104 for applying an oxidizing agent to a surface 106 is shown in FIGS. 1-7. The mobile device 104 may be any device movable across a surface. In an embodiment of the disclosed subject matter, the mobile device 104 is a trailer 108. The trailer 108 includes a frame 110 with wheels 112 allowing the trailer 108 to be moved about the surface 106. The frame 110 includes a connecting member 114 for connecting the trailer 108 to a vehicle (not shown) for moving the trailer 108 about the surface 106. In an embodiment, the connecting member 114 is a receiver for a ball-type hitch. In an embodiment, the surface 106 is turf of a natural or artificial nature.

Referring to FIGS. 1, and 4-7, the application system 202 is shown mounted to the trailer 108. The application system 202 includes an oxidizing agent system 302, and a liquid system 402.

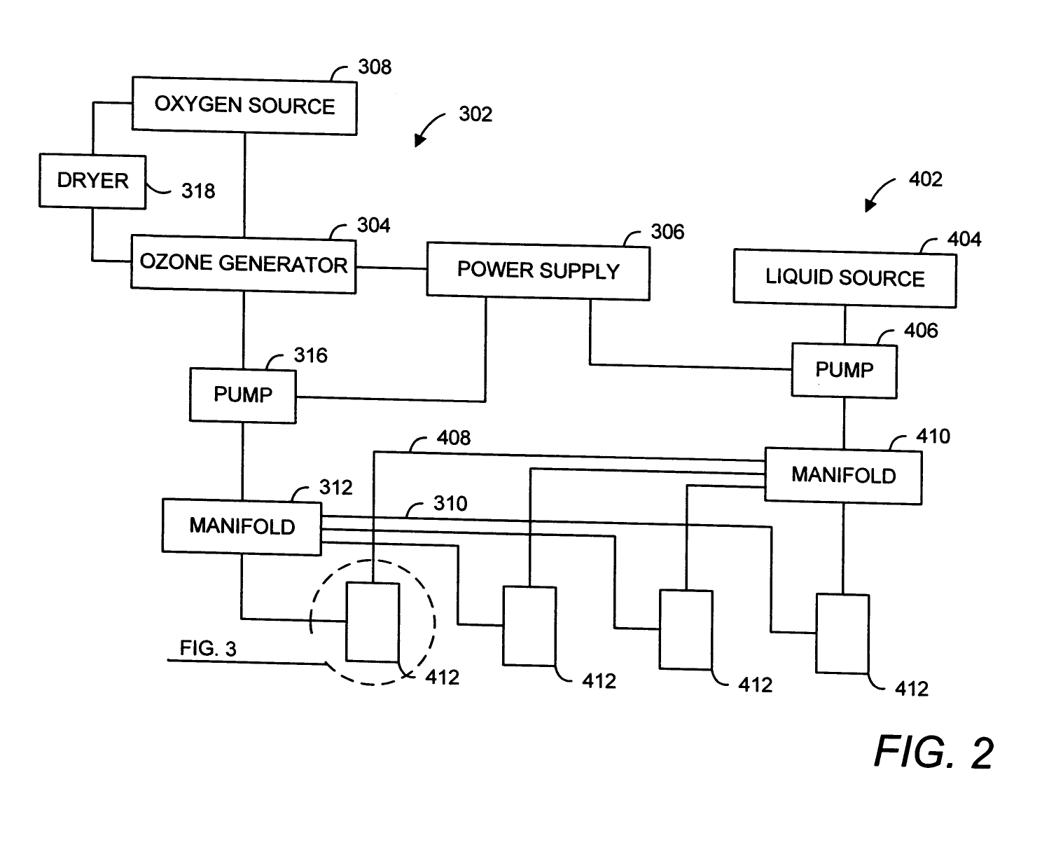

In an embodiment, the oxidizing agent system 302 is an ozone generator 304 operably connected to an power supply 306 providing electrical energy, and an oxygen source 308 (FIG. 2). The ozone generator 304 may be any ozone generator suitable for mounting on a mobile device 104, including the KH-CT5G ozone generating unit manufactured by DGOzone Ltd. of Shanghai, China. In an embodiment, the power supply 306 is a battery with an inverter providing AC power. In an embodiment, the power supply 306 includes electricity generated by a combustion engine connected to an electrical generator providing AC power. In an embodiment, the oxygen source 308 is ambient air from the environment containing the gaseous form of oxygen that is passed through a dryer 318 and then fed to the ozone generator 304 by a pump operably connected to the power supply 306. In another embodiment, the oxygen source 308 is the gaseous form of oxygen in the form of either liquefied oxygen or compressed oxygen gas that is bled to the ozone generator 304 by a regulator and valve. The oxygen containing material from the oxygen source 308 passes through the ozone generator 304, and the ozone generator 304 creates gaseous ozone 314 by passing an electrical current through material. The gaseous ozone 314 is transferred from the ozone generator 304 to the manifold 312, then to each nozzle 412 where the gaseous ozone 314 combines with the liquid 405. In an embodiment, the gaseous ozone 314 is transferred by a pump 316 at a pressure of about 60 pound per square inch (psi). The ozone generator 304 is operated by a user using conventional control mechanisms.

In an embodiment, the liquid system 402 includes a liquid source 404 connected to nozzles 412. In an embodiment, the liquid source 404 is a tank 414 containing water. A pump 406, operably connected to the power supply 306, moves the liquid 405 from the liquid source 404 to the nozzles 412 as a pressurized liquid by conduits 408 and a manifold 410. The nozzles 412 are arranged in an array to apply the liquid 405 to the surface 106 in a consistent manner. In an embodiment, the nozzles 412 are spaced along a support structure 116, such as a boom, at an end of the frame 110.

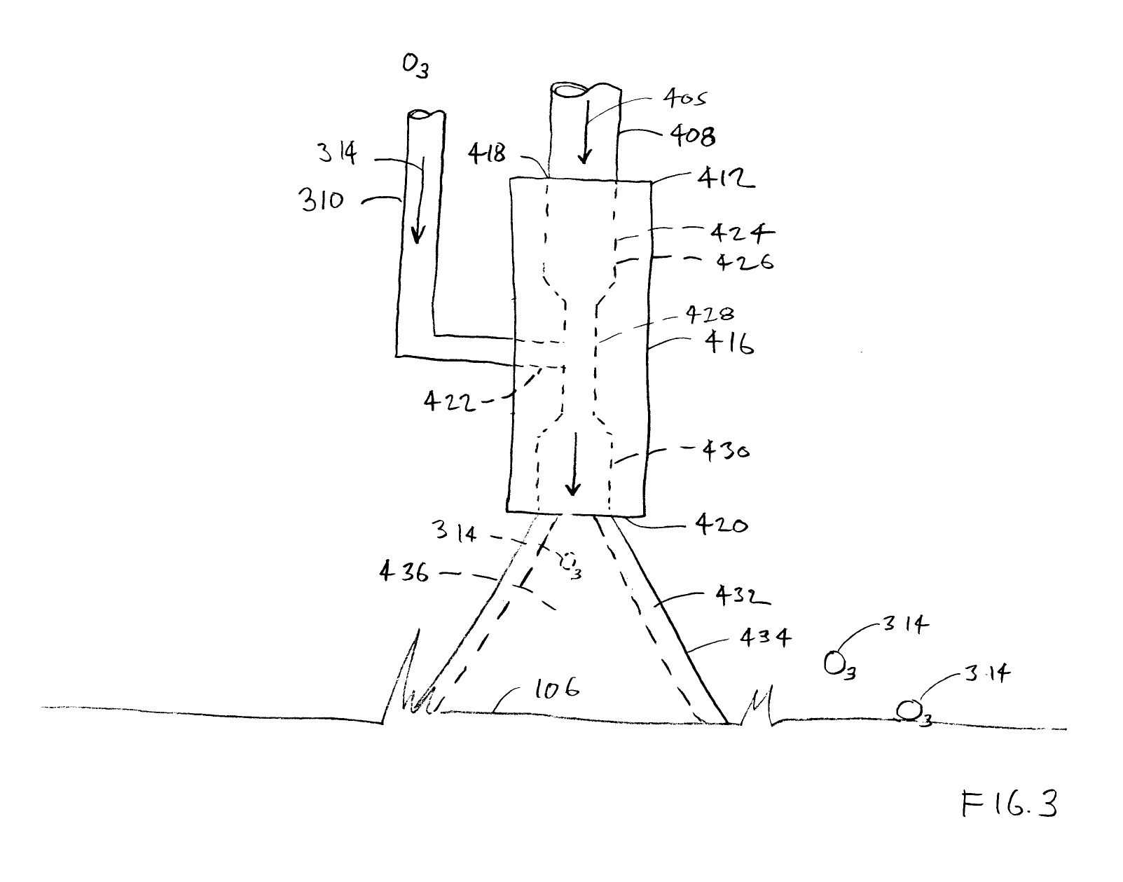

Referring to FIG. 3, the nozzles 412 include a body 416 defining a liquid flow passage 242 with an inlet 418 at one end and an outlet 420 at an opposite end. The liquid flow passage 424 has a wide diameter upper portion 426 immediately above a smaller diameter middle portion 428. A port 422 located between the inlet 418 and outlet 420 extends from the exterior of the nozzle 412 through the body 416 communicating with the middle portion 428.

Pressurized liquid 405 at a pressure of about 20 psi enters the liquid passage 424 through the inlet 418 via the conduit 408. Gaseous ozone 314 at about 60 psi enters the liquid passage 424 through the port 422 via the conduit 310. The venturi effect occurring at the middle portion 428 draws gaseous ozone 314 into the liquid 405 stream as it passes through the constriction mixing the gaseous ozone 314 with the liquid 405. A portion of the gaseous ozone 314 dissolves in the liquid 405 and the ozone-liquid mixture 432 exits the nozzle 412 through the outlet 420 and is applied to the surface 106. In an embodiment, the gaseous ozone 314 dissolved in the liquid 405 is about 0.6 milligrams per liter of liquid 405. The liquid system 402 is operated by a user using conventional control mechanisms.

By ozonation of the liquid 405, the ozone-liquid mixture 432 can be applied to the surface 106 and remain in contact with the surface 106. The ozone-liquid mixture 432 holds the dissolved gaseous ozone 314 in contact with the surface 106 and any pathogens thereon as the oxidizing agent disinfects the surface 106 by rendering any pathogens thereon ineffective. As oxidization of the pathogens occurs, the ozone reverts back to the gaseous form of oxygen leaving no harmful residue behind.

In an embodiment, the ozone-liquid mixture 432 exits the nozzle 412 in a conical spray pattern 434 as it is applied to the surface 106. In an embodiment, the conical spray pattern 434 is a solid cone of the ozone-liquid mixture 432. Applying the ozone-liquid mixture 432 to the surface 106 as the nozzles 112 move across the surface coats the surface 106 with the ozone-liquid mixture 432. I an embodiment, approximately 0.00162894 gallons of ozone-liquid mixture 432 is applied to each square foot of the surface 106.

In an embodiment, the conical spray pattern 434 is a hollow cone of the ozone-liquid mixture 432 with an open interior area 436. Within the interior area 136, undissolved gaseous ozone 314 is carried down to the surface 106.

In an embodiment, the ozone-liquid mixture 432 exits the nozzle 412 in a fan spray pattern as it is applied to the surface 106.

Gaseous ozone 314 that either comes out of the ozone-liquid mixture 432 or that is not dissolved in the liquid 405 in the nozzle 412 moves along with the ozone-liquid mixture 432 toward the surface 106 coating the surface 106 with a cloud of gaseous ozone 314.

The nozzles 412 are spaced apart from each other and positioned at a height above the surface 106 to achieve the desired spray coverage and/or overlap of spray on the surface 106. In an embodiment, the nozzles 412 are spaced and positioned to achieve an overlap of about five inches of spray coverage.

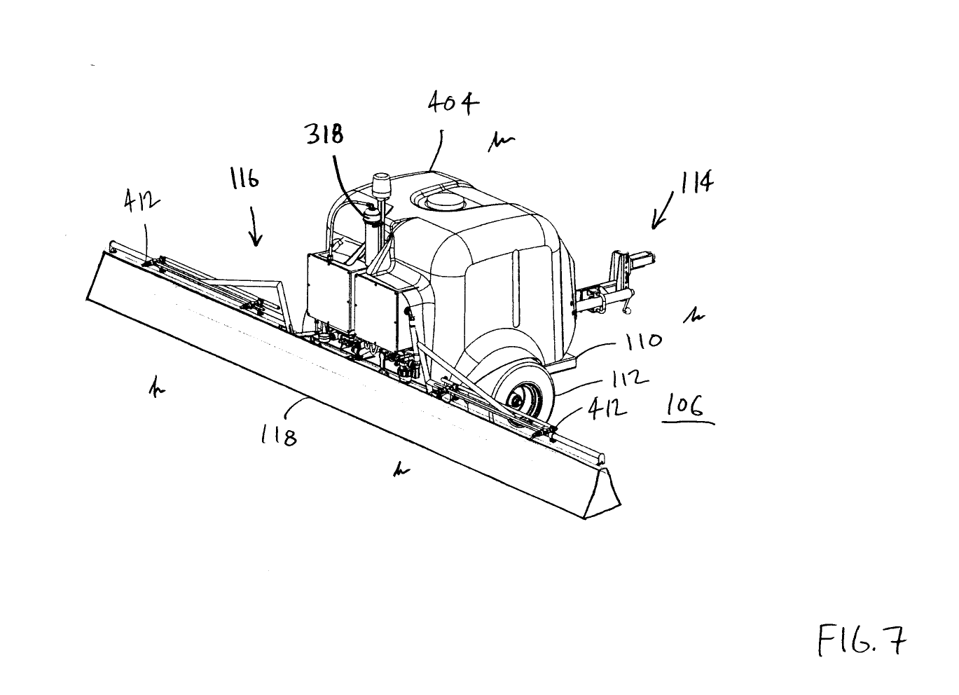

In an embodiment, a covering 118 extends downward and outward away from the nozzles 412 directing the ozone-liquid mixture 432 and cloud of gaseous ozone 314 downward after it is emitted from the nozzle 412 to minimize dispersion and dilution of the gaseous ozone 314 (FIG. 7). Directing the gaseous ozone 314 to the surface 106 allows the gaseous ozone 314 to be present in the air close to the ozone-liquid mixture 132 allowing the gaseous ozone 314 to diffuse into the ozone-liquid mixture 432 replacing the ozone that is consumed in the oxidative process. The covering 118 also confines the ozone-liquid mixture 432 and the undissolved gaseous ozone 314 close to the surface 106. The covering 118 may be manufactured from a rigid material, such as plastic, or may be manufactured from a flexible material, such as a water resistant fabric.

In an embodiment, a surfactant or wetting agent is added to the liquid 405 prior to the mixing of the gaseous ozone 314 with the liquid 405 in the nozzle 412 forming a surfactant-liquid mixture. The surfactant-liquid mixture enters the inlet 418 and gaseous ozone 314 is dissolved in the surfactant-liquid mixture, as described above with respect to the liquid 415, forming an ozone-surfactant-liquid mixture. The ozone-surfactant-liquid mixture exits the nozzle 412 through the outlet 420, as described above, and is applied to the surface 106. The addition of a surfactant to the liquid 405 decreases the tension of the liquid 405 increasing the amount of the ozone-liquid mixture in contact with the surface 106, and the dissolved ozone in contact with any pathogens on the surface 106. In an embodiment, the surfactant is the surfactant sold under the trademark BARDAC® LF-80 from Lonza, Inc. of Allendale, N.J.

In an embodiment, the application system 202 is mounted to a self-powered vehicle, such as a truck.

It is to be understood that while certain aspects of the disclosed subject matter have been shown and described, the disclosed subject matter is not limited thereto and encompasses various other embodiments and aspects.

Having thus described the disclosed subject matter, what is claimed as new and desired to be secured by Letters Patent is:

1. An oxidizing agent application device, comprising:
an oxidizing agent;
an application device, comprising:
a nozzle defining a passage extending between an inlet and an outlet;
a port communicating with the passage; and
wherein the oxidizing agent enters the passage through the port.

2. The application device of claim 1, wherein the oxidizing agent is gaseous ozone.

3. The application device of claim 2, further comprising:
a gaseous ozone generator; and
wherein the gaseous ozone is generated by the ozone generator.

4. The application device of claim 2, wherein the gaseous ozone enters the passage at a pressure of about 60 psi.

5. The application device of claim 2, further comprising:
a liquid; and
wherein the liquid enters the nozzle inlet.

6. The application device of claim 5, wherein the liquid enters the inlet at a pressure of about 20 psi.

7. The application device of claim 6, wherein:
the gaseous ozone is dissolved in the liquid within the nozzle forming an ozone-liquid mixture; and
the ozone-liquid mixture exits the nozzle through the outlet and is applied to a surface.

8. The application device of claim 7, wherein the ozone-liquid mixture includes about 0.6 milligrams of dissolved ozone per liter of liquid.

9. The application device of claim 7, further comprising:
a covering extending downward from the nozzle directing the ozone-liquid mixture toward the surface.

10. The application device of claim 5, further comprising:
a surfactant:
wherein the surfactant is added to the liquid forming a surfactant-liquid mixture; and
wherein the gaseous ozone is dissolved in the surfactant-liquid mixture within the nozzle forming an ozone-surfactant-liquid mixture; and
the ozone-surfactant-liquid mixture exits the nozzle through the outlet and is applied to a surface.

11. A method of applying an oxidizing agent on a surface, comprising:
providing an oxidizing agent;
providing an application device, comprising:
a nozzle defining a passage extending between an inlet and an outlet;
a port communicating with the passage; and
introducing the oxidizing agent into the passage through the port.

12. The method of claim 11, wherein the oxidizing agent is gaseous ozone.

13. The method of claim 12, further comprising:
providing a gaseous ozone generator; and
generating the gaseous ozone with the ozone generator.

14. The method of claim 12, wherein the gaseous ozone enters the inlet at a pressure of about 60 psi.

15. The method of claim 12, further comprising:
providing a liquid; and
introducing the liquid into the inlet.

16. The method of claim 15, wherein the liquid enters the inlet a pressure of about 20 psi.

17. The method of claim 16, wherein
the gaseous ozone is dissolved in the liquid within the nozzle forming an ozone-liquid mixture; and
expelling the ozone-liquid mixture from the nozzle; and
applying the ozone-liquid mixture to a surface.

18. The method of claim 17, wherein the ozone-liquid mixture includes about 0.6 milligrams of dissolved ozone per liter of liquid.

19. The method of claim 17, further comprising:
providing a covering extending downward from the nozzle directing the ozone-liquid mixture toward the surface.

20. The method of claim 15, further comprising:
providing a surfactant;
combining the surfactant with the liquid forming a surfactant-liquid mixture;
dissolving the gaseous ozone in the surfactant-liquid mixture within the nozzle forming an ozone-surfactant-liquid mixture;
expelling the ozone-surfactant-liquid mixture from the nozzle; and
applying the ozone-surfactant-liquid mixture to a surface.
